(19) United States
(12) Patent Application Publication
(10) Pub. No.: US 2004/0191221 A1
(43) Pub. Date: Sep. 30, 2004

(54) Methods of treating skeletal disorders using recombinant adeno-associated virus virions

(76) Inventors: Keiya Ozawa, Kawachi-gun (JP); Naomi Kobayashi, Yokohama City (JP); Tomoyuki Saito, Yokohama City (JP)

Correspondence Address: STOEL RIVES LLP, 201 SOUTH MAIN STREET, SUITE 1100, SALT LAKE CITY, UT, 84111, US

(21) Appl. No.: 10/152,238

(22) Filed: May 20, 2002

Related U.S. Application Data
(60) Provisional application No. 60/292,694, filed on May 22, 2001.

(51) Int. Cl. A01N 63/00; A01N 65/00; A61K 48/00; C12N 15/63; C12N 15/85; C12N 15/87

(52) U.S. Cl. 424/93.2; 424/93.1; 435/455

(57) ABSTRACT

Methods for delivering a heterologous gene to the periosteum or periosteal cells of a mammal by recombinant adeno-associated virus (rAAV) virions are described. The methods of the present invention are useful in the treatment of various skeletal disorders including degenerative diseases such as osteoarthritis and osteoporosis. The methods of the present invention are also useful in the treatment of inflammatory joint diseases such as rheumatoid arthritis. The heterologous gene can code for growth factors known to stimulate osteogenesis or chondrogenesis. Such growth factors include transforming growth factor-beta, insulin-like growth factor, fibroblast growth factor, and bone morphogenetic proteins. Alternatively, the heterologous gene can code for anti-inflammatory molecules such as tumor necrosis factor soluble receptor, interleukin-4, interleukin-10, and interleukin-13. The methods also allow for the rAAV virion delivery of genes coding for proteins known to inhibit osteoclast activity, thereby reducing bone resorption. An exemplary example of an osteoclast-inhibiting protein is osteoprotegerin.

### CROSS-REFERENCE TO RELATED APPLICATIONS

This application is related to provisional patent application Ser. No. 60/292,694, filed May 22, 2001 entitled “Gene Marking Study of Periosteum-Derived Cells Using Adeno-Associated Virus Vector” from which application priority is claimed under 35 USC §119(e)(1).

### FIELD OF THE INVENTION

The present invention relates generally to the delivery of adeno-associated virus vectors to skeletal tissue and cells, principally periosteal tissue and cells. More particularly, the present invention relates to gene therapy for the treatment of skeletal disorders, particularly those disorders involving articular cartilage and bone degradation.

### BACKGROUND

Skeletal disorders comprise a large group of heterogenous diseases affecting cells and tissues of bone, bone marrow, cartilage, tendons, and ligaments. Together, these diseases affect hundreds of millions of individuals worldwide, with a reported estimated treatment cost exceeding $500 billion ($215 billion in the United States) (Bone and Joint Decade Project, World Health Organization, website visited May 14, 2002 <http://www.boneandjointproject.org>). Globally, they are the largest cause of long-term disability and pain. Joint disease alone accounts for half of all chronic conditions in people aged 60 and older. Bone disorders are nearly as pervasive; over 40% of all women aged 50 and older experience a debilitating bone fracture related to osteoporosis (Bone and Joint Decade Project, supra). Current physical, surgical, and pharmacological therapeutic options are relatively ineffective in treating the most prevalent degenerative skeletal diseases (i.e., osteoarthritis, rheumatoid arthritis, and osteoporosis).

Osteoarthritis, also known as “osteoarthrosis” or “degenerative joint disease,” is the most common form of arthritis, affecting 12.1 percent of the U.S. adult population, i.e., 20.7 million people (Merck Co., ed. Osteoarthritis and neurogenic arthropathy. In: The Merck Manual of Diagnosis and Therapy. 17^{th} ed. Rathway, N.J.: Merck, (2002); Lawrence et al. (1998) Arthritis Rheum 41:778-799). It primarily afflicts the older population with approximately 25% of people over the age of 60 having significant pain and disability directly related to the disease; additionally, 50% of those over 60 years of age have radiologic signs of the disease, which demonstrates the widespread prevalence of osteoarthritis in this demographic cohort (Lawrence et al., supra).

In the U.S., osteoarthritis accounts for half of all chronic conditions in persons over 65 years of age, with one in eight persons having been clinically diagnosed with the disease (Lawrence et al., supra). At age 80, osteoarthritis is present in nearly every individual (Dequeker J, Dieppe P A, eds. Disorders of bone cartilage and connective tissue. In: Klippel J H, Dieppe P A, eds. Rheumatology. 2nd ed. London: Mosby, 1998). Moreover, the disease is responsible for the second highest cause of work loss, despite its overwhelming prevalence in retired populations (Lawrence et al., supra). Given these statistics, it is clear that the economic consequences of osteoarthritis are enormous.

Osteoarthritis causes pain in the joints without pervasive inflammation present. Minimal inflammatory involvement helps to characterize osteoarthritis from rheumatoid arthritis, whose principal clinical feature is insidious joint inflammation. Another distinguishing clinical feature of osteoarthritis is its restriction to articular cartilage; unlike patients suffering from rheumatoid arthritis, systemic symptoms are absent in patients having osteoarthritis. The pathologic features of osteoarthritis are focal ulceration and loss of articular cartilage with subsequent loss of joint function (due to the depletion of articular cartilage).

Articular cartilage has a very limited ability to regenerate itself as it lacks vasculature, innervation, and cartilage-producing cells. Any new articular cartilage that is endogenously produced (largely from migrating chondrocytes to the arthritic lesion) is insufficient to overcome the pathological loss. The disease is presently incurable.

In contrast to osteoarthritis, rheumatoid arthritis is an autoimmune disorder having systemic activity; however, the disease exerts its effects most strongly at the joints. An estimated 1% of the U.S. adult population is afflicted with rheumatoid arthritis, i.e., a patient population of approximately 2.1 million (Gabriel (2001) Rheum Dis Clin North Am 27:269-81; National Institute of Arthritis and Musculoskeletal and Skin Diseases statistics, website visited May 14, 2002, <http://www.niams.nih.gov>). The disease is characterized by a thickening of the synovial membrane resulting from a neutrophil and monocyte cell infiltrate and a subsequent cascade of pro-inflammatory cytokines and other inflammatory molecules. This inflammatory milieu drives synoviocyte hyperplasia and hypertrophy creating the characteristic enlarged synovial membrane, which then attaches to, invades, and erodes articular cartilage and subchondral bone. As the disease progresses, the cumulative degradation of the joint structures causes a disfigurement and ultimately a loss of joint function. Rheumatoid arthritis is also presently incurable.

Osteoporosis, the most common bone degenerative disorder, affects millions of individuals worldwide with treatment costs in the United States estimated at $10 billion annually. The disease is characterized by a gradual loss of bone mass that occurs as a function of age; this loss of bone mass significantly increases the risk of bone fractures (an estimated 20-25 million people in the United States are at increased risk for osteoporosis-related bone fractures). Existing pharmacological measures to treat or prevent osteoporosis focus on ensuring adequate calcium and vitamin D intake. Food and Drug Administration-approved therapies include hormone replacement therapy, selective estrogen receptor modulators, bisphosphonates, and calcitonin. All of these therapies have limited efficacy. If current demographic trends continue, the estimated population aged 50 and older in the United States is projected to double by 2020; without the development of effective therapies for osteoporosis, the projected costs of treatment are expected to increase by 2-3 fold.

Novel therapies under development for skeletal disorders include biological and physical methods for stimulating new bone (i.e., osteogenesis) and cartilage (i.e., chondrogenesis) formation at the sites of degradation or injury. The transplantation of bone marrow mesenchymal stem cells onto damaged areas of bone and cartilage as well as the injection of growth hormones into articular spaces has met with some success in stimulating osteogenic and chondrogenic processes (see, e.g., Minas et al. (1997) Orthopedics 20:525-538; Engkvist (1979) Scan J. Plast. Reconstr. Surg. 13:361-369; Rubak (1982) Acta Orthop. Scan. 53:181-186).

Stimulating a generalized chondrogenic process, however, may not be sufficient to effectively treat patients with articular cartilage defects (e.g., osteoarthritis and rheumatoid arthritis). Non-articular cartilage has been shown to rapidly degrade when present in the articular spaces, thereby limiting the grafting of other forms of cartilage where it is most needed to correct the joint defects caused by depleted articular cartilage. For example, in seeking novel treatments for osteoarthritis, surgeons have grafted fibrocartilage to the affected joint areas, but with little success due to the degradation of the graft. Consequently, significant effort has been directed toward finding ways to stimulate the formation of articular cartilage (as opposed to any other type of cartilage), since this is the endogenous form of cartilage found in the joints (which, presumably, will enable it to remain viable—at least longer than exogenous forms of grafted cartilage).

Articular cartilage covers the ends of all bones that form articulating joints. It is comprised of an extracellular matrix of collagen fibers (primarily Type II collagen) as well as a variety of proteoglycans. It has a hyaline appearance (i.e., it is clear, transparent, and granule-free), which distinguishes it morphologically from other forms of cartilage (e.g., fibrocartilage). Articular cartilage acts in the joint as a mechanism for force distribution and as a lubricant in the area of contact between the bones, whereas the proteoglycans impart compressibility. Without articular cartilage, stress concentration and friction would occur to such a degree that the joint would not permit ease of motion.

As mentioned above, natural cartilaginous repair mechanisms are limited in their ability to form new articular cartilage; whatever amount is formed is generally not adequate to replace severely damaged arthritic joint surfaces. Generally, since articular cartilage lacks both a blood supply and mesenchymal stem cells (i.e., cells able to differentiate into bone or cartilage-producing cells), it has a limited potential for self-repair.

Since articular cartilage in adults does not naturally regenerate to a significant degree once it has been destroyed, damaged adult articular cartilage has historically been treated by a variety of surgical interventions including replacement or excision (Minas et al., supra). With replacement or excision, regeneration of tissue may occur, although the tissue is usually temporary (especially if non-articular cartilage is used to replace damaged or lost articular cartilage) and inadequate to withstand the normal joint forces.

Replacing cartilaginous tissue with non-cartilaginous artificial materials generally has produced less than satisfactory results. Natural materials (such as resorbable materials) having characteristics similar to articular cartilage and suitable for constructing therapeutic matrices are currently unavailable. As mentioned above, fibrocartilage is rapidly degraded if grafted to articular surfaces. Because the opposing articular cartilage of mammalian joints is so fragile, it will not withstand abrasive interfaces that often result from the implantation of artificial cartilage. Additionally, joint forces are multiples of body weight that, in the case of the knee and hip, are typically encountered over a million cycles per year. Thus far, permanent artificial cartilage materials have not reflected natural articular cartilage properties, nor have they been able to be positioned securely enough to withstand such routine forces.

Biological means of stimulating articular formation have also been tried. For instance, chondrogenic factors have been used to stimulate cartilage formation in various models of arthritis. The use of chondrogenic factors, however, has generated somewhat disappointing results due primarily to the physical and chemical parameters that limit protein delivery in general: for example, the inability of proteins to be taken orally due to digestive breakdown, the necessity for systemic administration of potentially toxic concentrations of protein in order to achieve therapeutic concentrations at the target site, and the short half-life of proteins once at the target site necessitating frequent injections or continuous infusion.

Periosteal tissue is a promising new candidate in treating articular cartilage disorders. When grafted onto articular surfaces, periosteal tissue has been successful in generating new articular cartilage (O'Driscoll et al. (2001) Clin Orthop. 391S:S190-S207). Providing a growth factor, such as transforming growth factor-beta, with the periosteal graft has improved results. Periosteal tissue is derived from the periosteum, a fibrous membrane that covers bones except at their articular surfaces. It is comprised of fibrous connective tissue, proteoglycans, and periosteal cells. These cells possess osteogenic and chondrogenic activity, which has generated enthusiasm as a potential new way of treating bone and articular cartilage disorders.

The history of developing novel therapies for bone disorders is similar to that of the development of new articular cartilage therapies. As with chondrogenesis, a vast amount of research has been conducted to identify novel ways of stimulating bone repair. To date, these efforts have resulted in negligible success. The need for new bone repair therapies exists for repair of bone fractures, bone segmental defects, metastatic bone disease, and osteolytic bone disease (Oakes et al. (2000) Clin. Orthop. 379S:S101-S112). As mentioned above, also of great significance is osteoporosis in its various forms, including age-related osteoporosis, disuse osteoporosis, diabetes-related osteoporosis, glucocorticoid-related osteoporosis, and osteoporosis associated with post-menopausal hormone status. Other conditions characterized by the need for bone repair include primary and secondary hyperparathyroidism.

Bone morphogenetic proteins (BMPs), which belong to the transforming growth factor-beta superfamily, possess osteogenic and chondrogenic activity. They were first identified by Wozney J. et al. Science (1988) 242:1528-34, using gene cloning techniques, following earlier descriptions characterizing the biological activity in extracts of demineralized bone (Urist M. Science (1965) 150:893-99). They are expressed by normal osteoblasts, and stimulate bone formation in vivo. This property suggests the potential usefulness of BMPs as therapeutic agents in various skeletal disorders.

Although BMPs and other growth factors possess osteogenic and chondrogenic activity, there are certain disadvantages to using them as therapeutic agents. For instance, growth factors (including BMPs) and their receptors are expressed in a large variety of non-skeletal tissue, which suggests that they may have pleiotropic effects, increasing the probability for adverse or unwanted reactions in response to systemic administration. If administered non-systemically, e.g, by direct injection into the joints, BMPs and other growth factors are subject to proteolytic cleavage and degradation, complicating the goal of maintaining a sustained therapeutic dose where it is most needed.

Gene therapy methods can potentially overcome the problems associated with systemic delivery of growth factors by delivering the genes coding for these proteins directly to the site where they are needed. Once delivered, sustained expression can be achieved with the use of a constitutive gene promoter. Alternatively, selective expression can be achieved with the use of a cell or tissue-specific gene promoter or inducible expression with the use of an inducible promoter.

Genes may be delivered to a patient in a variety of ways. There are transfection methods, including chemical methods such as calcium phosphate precipitation and liposome-mediated transfection, and physical methods such as electroporation. Current viral-mediated gene delivery vectors include those based on retrovirus, adenovirus, herpes virus, pox virus, and adeno-associated virus (AAV).

### Adeno-Associated Virus-Mediated Gene Therapy

Adeno-associated virus, a parvovirus belonging to the genus Dependovirus with six known serotypes (designated AAV-1 through AAV-6), has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing a gene of interest, a suitable host cell line is transfected with an AAV vector containing the gene, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the therapeutic gene. The gene of interest is then replicated and packaged as though it were a wt AAV genome, forming a recombinant AAV virion. When a patient's cells are transduced with the resulting rAAV virion, the gene enters and is expressed in the patient's cells. Because the patient's cells lack the rep and cap genes, as well as the accessory function genes, the rAAV virion cannot further replicate and package its genomes. Moroever, without a source of rep and cap genes, wt AAV virions cannot be formed in the patient's cells.

It would be a significant advancement in the art to develop strategies for treating skeletal disorders that allow for the administration of osteogenic and/or chondrogenic growth factors without eliciting unwanted side effects associated with systemic delivery. Such methods are disclosed herein.

### SUMMARY

The methods of the present invention provide for the delivery of one or more heterologous genes to periosteal cells and tissue using adeno-associated virus (rAAV) virions. In one embodiment, periosteal cells and tissue are transduced by rAAV virions ex vivo. Once the periosteal cells or tissue are transduced ex vivo, the cells and/or tissue are then grafted onto a mammal, preferably at or near the articular surfaces, where the heterologous gene is expressed. The periosteal cells and/or tissue can be derived from the mammal receiving the periosteal graft. Alternatively, the periosteal cells and/or tissue can be derived from another source than the mammal receiving the periosteal graft. The periosteal cells can be embedded in a matrix, preferably collagen, most preferably type II collagen, or they can be grafted directly onto the target site. Once grafted, the periosteal cells can be anchored in place by the use of a periosteum-derived patch.

In another embodiment, periosteal cells and tissue are transduced by rAAV virions in vivo. Recombinant AAV virions can be injected directly into the periosteum of a mammal by passing a needle through the skin and underlying tissue and making direct contact with the periosteum. Alternatively, a surgical incision can be made in the integument to expose the periosteum of a mammal. Once exposed, the rAAV virions can be directly injected into the periosteum. In another embodiment, the rAAV virions are delivered by irrigating the exposed periosteum in a solution containing the rAAV virions.

In another embodiment, the periosteal cells and tissue are transduced by rAAV virions using a combination of an in vivo and an ex vivo approach.

In one embodiment, recombinant AAV virions are used to deliver genes having chondrogenic activity (i.e., genes coding for proteins that stimulate cartilage formation) to the periosteum and/or periosteal cells of a mammal, preferably a human. Preferably, the chondrogenic genes are delivered to treat an articular cartilage defect in a mammal having an articular cartilage defect. In a preferred embodiment, the articular cartilage defect is rheumatoid arthritis. In an especially preferred embodiment, the articular cartilage defect is osteoarthritis.

In yet another embodiment, recombinant AAV virions are used to deliver genes having osteogenic activity (i.e., genes coding for proteins that stimulate bone formation) to the periosteum and/or periosteal cells of a mammal. In a preferred embodiment, the osteogenic genes are delivered to treat bone disorders. In an especially preferred embodiment, the bone disorder is osteoporosis.

In certain embodiments, rAAV virions are used to deliver one or more genes encoding one or more growth factors to the periosteum and/or periosteal cells of a mammal, preferably a human. The growth factors may be one or more of the bone morphogenetic proteins; one or more of the fibroblast growth factor proteins; one or more of the transforming growth factor-beta proteins; or one or more of the insulin-like growth factor proteins.

In still other embodiments, rAAV virions are used to deliver one or more genes encoding one or more anti-inflammatory molecules to the periosteum and/or periosteal cells of a mammal, preferably a human. For example, the anti-inflammatory molecule may be interleukin-1 receptor antagonist; interleukin-1 receptor; interleukin-1 soluble receptor; tumor necrosis factor soluble receptor; tumor necrosis factor receptor; interferon-alpha; interleukin-4; interleukin-10; or interleukin-13.

In yet another embodiment, rAAV virions are used to deliver one or more genes encoding a protein or proteins that inhibit osteoclasts. In a preferred embodiment, the osteoclast inhibitor is osteoprotegerin.

### BRIEF DESCRIPTION OF THE DRAWINGS

FIG. 1. Photomicrograph of periosteum-derived cells on culture plate stained with 5-bromo-4-chloro-3-indolyl-beta-D-galactoside (X-gal). X-gal staining was performed 3 days (A), 1 (B), 2 (C), 4 (D), and 12 (E) weeks after rAAV-LacZ transduction. Arrows indicate LacZ-positive cells (magnification ×100). Panel F shows non-transduced control cells.

FIG. 2. Photomicrograph of collagen gel including periosteal cells stained with X-gal and hematoxin-eosin. Periosteal cells in collagen gel were cultured for 3 days (A), 1 (B), 2 (C), and 4 (D) weeks after rAAV-LacZ transduction. Arrows indicate LacZ-positive cells (magnification ×40).

FIG. 3. Photomicrograph of rabbit knee tissue transplanted with collagen gel containing periosteal cells. Periosteal cells grew in the transplanted site and expressed LacZ under the periosteum patch 1 (A) and 2 (B) weeks after transplantation. Open arrows indicate periosteum patch and closed arrows indicate LacZ-positive periosteum-derived cells. LacZ-positive cells were not detected in cell-free collagen transplants (C) and non-transduced periosteal cells in collagen transplants (D).

### DETAILED DESCRIPTION

The present invention embraces the use of recombinant adeno-associated virus (rAAV) virions to deliver one or more heterologous genes to the periosteum or periosteal cells of a mammal. By “periosteum” is meant the membrane of fibrous connective tissue that closely invests all bones except at the articular surfaces. By “periosteal cells” is meant cells derived exclusively from the periosteum. Periosteal cells can be separated from the periosteum by well-known techniques in the art; subjecting periosteal tissue to trypsinization is but one of many examples for obtaining periosteal cells. The cells, once released from the periosteum or periosteal tissue, can then be grown in cell culture.

In the context of the present invention, a “recombinant AAV virion” or “rAAV virion” is an infectious virus composed of an AAV protein shell (i.e., a capsid) encapsulating a “recombinant AAV (rAAV) vector,” the rAAV vector defined herein as comprising a heterologous nucleic acid molecule and one or more AAV inverted terminal repeats (ITRs). By “heterologous” is meant a nucleic acid molecule flanked by nucleotide sequences not found in association with the nucleic acid molecule in nature. Alternatively, “heterologous” embraces the concept of a nucleic acid molecule that itself is not found in nature (e.g., synthetic sequences having codons different from a native gene). Allelic variation or naturally occurring mutational events do not give rise to heterologous nucleic acid molecules, as used herein. Heterologous nucleic acid molecules can be in the form of genes, promoters, enhancers, or any other nucleic acid-containing molecule so long as they adhere to the definition of “heterologous,” as used herein. The heterologous nucleic acid molecule can be incorporated into a rAAV vector using standard molecular biological techniques that are well known to the skilled artisan.

Recombinant AAV vectors can be constructed using recombinant techniques that are known in the art and include one or more heterologous genes flanked by functional ITRs. The ITRs of the rAAV vector need not be the wild-type nucleotide sequences, and may be altered, e.g., by the insertion, deletion, or substitution of nucleotides, so long as the sequences provide for proper function, i.e., rescue, replication, and packaging of the AAV genome.

Recombinant AAV virions may be produced using a variety of art-recognized techniques. For example, the skilled artisan can use wt AAV and helper viruses to provide the necessary replicative functions for producing rAAV virions (see, e.g., U.S. Pat. No. 5,139,941, herein incorporated by reference). Alternatively, a plasmid, containing helper function genes, in combination with infection by one of the well-known helper viruses can be used as the source of replicative functions (see e.g., U.S. Pat. No. 5,622,856, herein incorporated by reference; U.S. Pat. No. 5,139,941, supra). Similarly, the skilled artisan can make use of a plasmid, containing accessory function genes, in combination with infection by wt AAV, to provide the necessary replicative functions. As is familiar to one of skill in the art, these three approaches, when used in combination with a rAAV vector, are each sufficient to produce rAAV virions. Other approaches, well known in the art, can also be employed by the skilled artisan to produce rAAV virions.

In a preferred embodiment of the present invention, the triple transfection method (described in detail in U.S. Pat. No. 6,001,650, the entirety of which is incorporated by reference) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV vector. One of skill in the art will appreciate, however, that the nucleic acid sequences encoded by these vectors can be provided on two or more vectors in various combinations. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The AAV helper function vector encodes the “AAV helper function” sequences (i.e., rep and cap), which function in trans for productive AAV replication and encapsidation. Preferably, the AAV helper function vector supports efficient AAV vector production without generating any detectable wt AAV virions (i.e., AAV virions containing functional rep and cap genes). An example of such a vector, pHLP19 is described in U.S. Pat. No. 6,001,650, supra, and in Example 1, infra. The rep and cap genes of the AAV helper function vector can be derived from any of the known AAV serotypes. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno1 is used (details regarding pLadeno1 are described in U.S. Pat. No. 6,004,797, herein incorporated by reference in its entirety). This plasmid provides a complete set of adenovirus accessory functions for AAV vector production, but lacks the components necessary to form replication-competent adenovirus.

The rAAV vector can be a vector derived from any AAV serotype, including without limitation, AAV-1, AAV-2, AAV-3A, AAV-3B, AAV-4, AAV-5, AAV-6, etc. AAV vectors can have one or more of the wt AAV genes deleted in whole or in part, i.e., the rep and/or cap genes, but retain at least one functional flanking ITR sequence, as necessary for the rescue, replication, and packaging of the AAV virion. Thus, an AAV vector is defined herein to include at least those sequences required in cis for viral replication and packaging (e.g., functional ITRs). The ITRs need not be the wild-type nucleotide sequences, and may be altered, e.g., by the insertion, deletion, or substitution of nucleotides, so long as the sequences provide for functional rescue, replication, and packaging. AAV vectors can be constructed using recombinant techniques that are known in the art to include one or more heterologous genes flanked with functional AAV ITRs.

The heterologous gene is operably linked to a heterologous promoter (constitutive, cell-specific, or inducible) such that the gene is capable of being expressed in the patient's target cells under appropriate or desirable conditions. By “operably linked” is meant an arrangement of elements wherein the components so described are configured so as to perform their usual function. Thus, control sequences operably linked to a coding sequence are capable of effecting the transcription of the coding sequence. The control sequences need not be contiguous with the coding sequence, so long as they function to direct the transcription thereof. Thus, for example, intervening untranslated yet transcribed sequences can be present between a promoter sequence and the coding sequence and the promoter sequence can still be considered “operably linked” to the coding sequence.

Numerous examples of constitutive, cell-specific, and inducible promoters are known in the art, and one of skill could readily select a promoter for a specific intended use, e.g., the selection of the osteocalcin gene promoter for osteoprogenitor cell-specific gene expression (Cancela et al. (1990) J Biol Chem 265:15040-15048; Lian et al. (2000) Clin Orthop. 379S:S146-S155; GenBank Accession No. NM_{—}00071 1), the selection of the aggrecan gene promoter (Doege et al. (2002) J Biol Chem 277:13989-13997; GenBank Accession No. AF031586) or the cartilage oligomeric protein gene promoter (GenBank Accession No. AF069520) for chondrocyte-specific gene expression, the selection of the constitutive CMV promoter for strong levels of continuous or near-continuous expression, or the selection of the inducible ecdysone promoter for induced expression. Induced expression allows the skilled artisan to control the amount of protein that is synthesized. In this manner, it is possible to vary the concentration of therapeutic product. Other examples of well known inducible promoters include: steroid promoters (e.g., estrogen and androgen promoters) and metallothionein promoters.

Gene expression can be enhanced by way of an “enhancer element.” By “enhancer element” is meant a DNA sequence (i.e., a cis-acting element) that, when bound by a transcription factor, increases expression of a gene relative to expression from a promoter alone. There are many enhancer elements known in the art, and the skilled artisan can readily select an enhancer element for a specific purpose. For example, to enhance chondrocyte-specific gene expression, the skilled artisan can make use of the recently identified cartilage oligomeric matrix protein gene enhancers (Issack et al. (2000) J Orthop Res 18:345-350).

The delivery of a heterologous gene or genes to the periosteum or periosteal cells of a mammal by rAAV virions can serve many purposes, including the introduction of a marker gene into periosteal cells or tissue to facilitate diagnostic procedures. For example, a clinician or other medical practitioner may desire to determine the number of viable periosteal cells in a segment of periosteal tissue to better diagnose the extent of a cartilage or bone disorder. By transducing periosteal tissue with rAAV virions containing one or more marker genes, the clinician will be able to quantify viable periosteal cells. Since the ability to establish long-term heterologous gene expression is characteristic of AAV, the clinician may find it especially useful to employ the methods of the present invention in conducting gene marker studies in periosteal cells.

The skilled artisan may wish to make use of the present invention to deliver genes to the periosteum or periosteal cells using rAAV virions to establish their function (a “functional genomics” approach). For example, a researcher may wish to evaluate the function of chondrocyte and/or osteoblast-specific genes in periosteal cells by delivering the chondrocyte and/or osteoblast-specific genes to the periosteum (either by using an in vivo, ex vivo, or an in vivo+ex vivo approach) using rAAV virions. The gene products can be localized to the cytosol of a periosteal cell or cells, embedded across or adjacent to a lipid membrane (plasma and/or organelle), or secreted. By using the methods of the present invention, the skilled artisan can over-express a gene of unknown function or, alternatively, express anti-sense mRNA to establish gene function. In this manner, by expressing the rAAV virion-delivered genes in the periosteal cells, the skilled artisan, by using the teachings of the present invention, can determine the function of the gene product (a “functional proteomics” approach).

The present invention preferably embraces the delivery of rAAV virions comprising heterologous genes that, when expressed in a mammal having a skeletal disorder, facilitate the growth and/or repair of bone and/or cartilage in order to treat the skeletal disorder. By “skeletal disorder” is meant any disease or injury that causes reduced function in a component of the skeletal system and/or its associated connective tissue. A bone fracture or a reduction in bone mass due to osteoporosis, or a reduction in articular cartilage due to osteoarthritis or rheumatoid arthritis, or a bone fracture or a reduction in articular cartilage due to injury are examples of skeletal disorders in the context of the present invention. Thus, the invention includes the delivery of genes comprising DNA sequences that code for one or more peptides, polypeptides, or proteins that stimulate bone and/or cartilage synthesis, which are useful for the treatment of skeletal injuries or diseases, such genes including, but not limited to: DNA encoding any of the epidermal growth factor proteins; DNA encoding platelet derived growth factor; and DNA encoding cartilage-derived morphogenic protein.

The invention also includes the delivery of genes comprising DNA sequences that code for one or more peptides, polypeptides, or proteins that inhibit inflammation, which may be especially useful in the treatment of rheumatoid arthritis but may also find use in the treatment of osteoarthritis and other skeletal disorders, such genes including, but not limited to: DNA encoding interleukin 1 receptor antagonist; DNA encoding interleukin 1 soluble receptor; DNA encoding tumor necrosis factor soluble receptor; DNA encoding tumor necrosis factor receptor; DNA encoding interleukin-4; DNA encoding interleukin-10; DNA encoding interleukin-13; DNA encoding interferon alpha; and DNA encoding interleukin-2 (which may be of benefit in treating bone tumors).

The invention also includes the delivery of genes comprising DNA sequences that code for one or more peptides, polypeptides, or proteins that inhibit osteoclast activity, which may be useful in the treatment of osteporosis and other bone-loss disorders, such genes including, but not limited to: DNA encoding interferon-beta for the inhibition of osteoclast differentiation; and DNA encoding osteoclast inhibitory peptide.

Periosteal tissue or periosteal cells can be transduced by rAAV virions using in vivo methods, ex vivo methods, or a combination of the two methods. Periosteal cells can be transduced ex vivo, for example, by taking harvested periosteal tissue and either transducing periosteal tissue itself while the tissue is in tissue culture, or transducing periosteal cells (which have been isolated from periosteal tissue) while the periosteal cells are growing in cell culture. Once the periosteal tissue or periosteal cells have been transduced by the rAAV virions, the transduced material can be grafted onto an organism from which the periosteal material was not obtained (an allograft) or back onto the organism from which the periosteal material was obtained (an autograft). The transduced periosteal tissue or cells can be directly grafted onto an organism or placed within a scaffold structure prior to grafting onto the host (Minas et al., supra; van den Berg et al. (2001) Clin Orthop. 391S:S244-S250). Scaffolding material can be comprised of any suitably inert material having the proper structural integrity to support a periosteal graft. Such material can include collagen (particularly type II collagen), plastic, sintered hydroxyapatite, bioglass, aluminates, ceramics, and the like. Such material may, but need not be, biodegradable. In addition to, or in lieu of, scaffolding, periosteal material can also be superimposed with a periosteal patch to help anchor the graft onto the appropriate site within the host (see Example 5, infra).

In vivo transduction can be accomplished by way of direct injection of rAAV virions through the skin and underlying layers into the periosteal tissue or by surgical access, isolation of the periosteum, and administration of rAAV virions. Both techniques are well known in the art.

It is an exemplary feature of the present invention to provide methods for stimulating bone and/or cartilage formation (i.e., osteogenesis and chondrogenesis) to treat a skeletal disorder such as osteoporosis, osteoarthritis, or rheumatoid arthritis. Several growth factors have been shown to have osteogenic and/or chondrogenic potential. Members of the transforming growth factor beta superfamily, including bone morphogenetic proteins, are exemplary examples of growth factors having such activity.

In one preferred embodiment, the rAAV virions are used to deliver one or more of the transforming growth factor-beta (TGF-β) genes to a mammal having a skeletal disorder, particularly an articular cartilage disorder. There are at least three known human TGF-β genes: TGF-β1 (GenBank Accession No. XM_{—}008912); TGF-β2 (GenBank Accession No. XM_{—}001754); and TGF-β3 (GenBank Accession No. NM_{—}003239).

In another embodiment, rAAV virions are used to deliver one or more of the bone morphogenetic protein (BMP) genes. There are several known human BMP genes, with BMP-2 through BMP-8 having strong osteogenic and chondrogenic effects. The sequences for the BMP genes are known, with some having multiple splice variants (as reflected in multiple GenBank entries): BMP-1 (GenBank Accession No. M22488); BMP-2 (GenBank Accession Nos. NM_{—}001200 and AF040249); BMP-3 (GenBank Accession No. NM_{—}001201); BMP-4 (GenBank Accession Nos. NM_{—}130850, NM_{—}13851, NM_{—}001202, and BC020546); BMP-5 (GenBank Accession Nos. NM_{—}021073 and M60314); BMP-6 (GenBank Accession No. NM_{—}001718); BMP-7 (GenBank Accession Nos. NM_{—}001719, XM_{—}030621 and BC008584); BMP-8 (GenBank Accession No. XM_{—}001720); BMP-9 (GenBank Accession No. AF188285); BMP-10 (GenBank Accession Nos. NM_{—}014482 and AF101441); and BMP-1 1 (GenBank Accession No. AF100907).

In an alternative embodiment of the present invention, the rAAV virions comprise one or more of the IGF genes. In addition to members of the transforming growth factor beta superfamily, exemplary examples of osteogenic and chondrogenic factors include members of the insulin-like growth factor (IGF) family. There are several known human IGF genes with two published in GenBank: IGF-1 (GenBank Accession No. NM_{—}000618) and IGF-2 (GenBank Accession No. NM_{—}000612).

In yet another embodiment, the rAAV virions comprise one or more of the fibroblast growth factor (FGF) genes. Members of the fibroblast growth factor family are also exemplary examples of proteins having osteogenic and chondrogenic activity; the clinician therefore, using the methods of the present invention, will find use for genes encoding any of the FGF proteins in treating patients with skeletal disorders. Numerous human FGF genes have been identified, several with transcriptional variants (reflected in several GenBank entries): FGF-1 (GenBank Accession Nos. XM_{—}054732, NM_{—}000800, NM_{—}033136, and NM_{—}033137); FGF-2 (GenBank Accession Nos. XM_{—}055784 and NM_{—}002006); FGF-3 (GenBank Accession No. NM_{—}005247); FGF-4 (GenBank Accession Nos. XM_{—}053627 and NM_{—}002007); FGF-5 (GenBank Accession Nos. XM_{—}003444, NM_{—}010203, NM_{—}033143, and NM 004464); FGF-6 (GenBank Accession No. NM_{—}020996); FGF-7 (GenBank Accession No. XM_{—}017651); FGF-8 (GenBank Accession No. AH006649); FGF-9 (GenBank Accession No. NM_{—}002010); FGF-1 1 (GenBank Accession No. AY094623); FGF-17 (GenBank Accession No. AF497475); FGF-18 (GenBank Accession Nos. NM_{—}033649 and NM_{—}003862); FGF-19 (GenBank Accession Nos. AF110400 and NM_{—}005117); and FGF-23 (GenBank Accession No. NM_{—}020638).

In another embodiment of the present invention, rAAV virions can be used to deliver genes encoding osteoclast inhibitors to prevent or reduce bone resorption, osteoprotegerin being an exemplary example. The sequence for osteoprotegerin is published (GenBank Accession Nos. NM_{—}002546 and U94332).

The recombinant AAV virion-delivered heterologous gene or genes (i.e., genes encoding growth factors, anti-inflammatory molecules, anti-inflammatory cytokines, inhibitors of osteoclast activity, etc.) are expressed in periosteal cells or tissue at a level sufficient to achieve a therapeutic effect. By “therapeutic effect” is meant a level sufficient to stimulate osteogenesis and/or chondrogenesis so that a clinical sign or symptom of a skeletal disorder is ameliorated. For example, in a patient suffering from rheumatoid arthritis, the delivered genes are expressed at a level such that the clinical signs and symptoms of inflammation in the joint are reduced (e.g., a reduction in synovial thickness, a reduction in the articular concentration of pro-inflammatory molecules, etc.) and/or an increase in articular cartilage is observed.

The dose of rAAV virions required for delivery of one or more heterologous genes to a periosteal cell to achieve a particular therapeutic effect, e.g., the units of dose in viral genomes (vg)/per mammal or vg/kilogram of body weight (vg/kg), will vary based on several factors including: the level of gene expression required to achieve a therapeutic effect, the specific skeletal disorder being treated, a potential host immune response to the rAAV virion (e.g., for in vivo delivery), a host immune response to the gene product, the area to be treated, the mode of treatment (i.e., in vivo or ex vivo), and the stability of the gene product. A rAAV virion dose may vary between 1×10⁶ vg/kg to 1×10¹³ vg/kg or even higher; the optimal dose can be readily ascertained by the skilled artisan during pre-clinical experimentation, clinical trials, and the like.

In the context of dose, the term “viral genome” is synonymous with “virion,” as a viral genome comprises the rAAV vector (containing the gene that is delivered to and expressed in the mammal), the rAAV vector being encapsulated in the rAAV virion. As those skilled in the art are well aware, when referring to dose, viral genome is the preferred term as quantitative measurements for dose have as their endpoint the detection of viral genomes. Several such quantitative measurements are well known in the art including, but not limited to, the dot blot hybridization method (described in U.S. Pat. No. 6,335,011, herein incorporated by reference) and the quantitative polymerase chain reaction (QPCR) method (described in Real Time Quantitative PCR. Heid C. A., Stevens J., Livak K. J., and Williams P. M. 1996. Genome Research 6:986-994. Cold Spring Harbor Laboratory Press). As mentioned above, the skilled artisan can readily determine a rAAV virion dose range to treat a patient having a particular skeletal disorder based on the aforementioned factors, as well as other factors that are well known in the art.

By using the methods of the present invention, rAAV virions comprising a heterologous gene were shown to transduce cultured periosteal cells.

The following examples are presented in order to more fully illustrate the preferred embodiments of the invention. They should in no way be construed, however, as limiting the broad scope of the invention, which is solely limited by the appended claims.

### EXAMPLE 1

### RECOMBINANT AAV-BETA GALACTOSIDASE (LacZ)

### VIRION PREPARATION

Recombinant AAV virions containing the beta galactosidase (LacZ) gene were prepared using a triple-transfection procedure described in U.S. Pat. No. 6,001,650, supra.

### Vector Construction

### AAV pHLP19 Helper Function Vector Construction

The AAV pHLP19 helper function vector was constructed using standard molecular biological techniques; its construction is described in detail in U.S. Pat. No. 6,001,650, supra.

To summarize, the AAV pHLP19 helper function vector was constructed in a several-step process using AAV-2 sequences derived from the AAV-2 provirus, pSM620, GenBank Accession Numbers K01624 and K01625. First, the ITRs were removed from the rep and cap sequences. Plasmid pSM620 was digested with SmaI and PvuII, and the 4543 bp rep-and cap-encoding SmaI fragment was cloned into the SmaI site of pUC19 to produce the 7705-bp plasmid, pUCrepcap. The remaining ITR sequence flanking the rep and cap genes was then deleted by oligonucleotide-directed mutagenesis using the oligonucleotides 145A (5′-GCTCGGTACCCGGGCGGAGGGGTGGAGTCG-3′) (SEQ ID NO: 1) and 145B (5′-TAATCATTAACTACAGCCCGGGGATCCTCT-3′) (SEQ ID NO:2). The resulting plasmid, pUCRepCapMutated (pUCRCM) (7559 bp) contains the entire AAV-2 genome (AAV-2 genome, GenBank Accession Number NC_{—}001401) without any ITR sequence (4389 bp). SrfI sites, in part introduced by the mutagenic oligonucleotides, flank the rep and cap genes in this construct. The AAV sequences correspond to AAV-2 positions 146-4,534.

Second, an Eco47III restriction enzyme site was introduced at the 3′ border of p5. This Eco47III site was introduced at the 3′ end of the p5 promoter in order to facilitate excision of the p5 promoter sequences. To do this, pUCRCM was mutagenized with primer P547 (5′-GGTTTGAACGAGCGCTCGCCATGC-3′) (SEQ ID NO:3). The resulting 7559 bp plasmid was called pUCRCM47III.

Third, an assembly plasmid, called pBluntscript, was constructed. The polylinker of pBSII SK+ was changed by excision of the original with BssHII and replaced with oligonucleotides blunt 1 and 2. The resulting plasmid, pBluntscript, is 2830 bp in length, and the new polylinker encodes the restriction sites EcoRV, HpaI, SrfI, PmeI, and Eco47III. The blunt 1 sequence is 5′-CGCGCCGATATCGTTAACGCCCGGGCGTTTAAACAGCGCTGG-3′ (SEQ ID NO:4) and the blunt 2 sequence is 5′-CGCGCCAGCGCTGTTTAAACGCCCGGGCGTTAACGATATCGG-3′ (SEQ ID NO:5).

Fourth, the plasmid pH1 was constructed by ligating the 4397 bp rep-and cap-encoding SmaI fragment from pUCRCM into the SrfI site of pBluntscript, such that the HpaI site was proximal to the rep gene. Plasmid pH1 is 7228 bp in length. Fifth, the plasmid pH2 was constructed. Plasmid pH2 is identical to pH1 except that the p5 promoter of pH1 was replaced by the 5′ untranslated region of pGN1909 (ATCC Accession Number 69871). Plasmid pGN1909 construction is described in detail in U.S. Pat. No. 5,622,856, herein incorporated by reference in its entirety). To accomplish this, the 329 bp AscI(blunt)-SfiI fragment encoding the 5′ untranslated region from pW19091acZ (described in detail in U.S. Pat. No. 5,622,856, supra) was ligated into the 6831 bp SmaI(partial)-SfiI fragment of pH1, creating pH2. Plasmid pH2 is 7155 bp in length.

Sixth, pH8 was constructed. A p5 promoter was added to the 3′ end of pH2 by insertion of the 172 bp, SmaI-Eco47III fragment encoding the p5 promoter from pUCRCM47III into the Eco47III site in pH2. This fragment was oriented such that the direction of transcription of all three AAV promoters are the same. This construct is 7327 bp in length.

Seventh, the AAV helper function vector pHLP19 was constructed. The TATA box of the 3′ p5 (AAV-2 positions 255-261, sequence TATTTAA (SEQ ID NO:6)) was eliminated by changing the sequence to GGGGGGG (SEQ ID NO:7) using the mutagenic oligonucleotide 5DIVE2 (5′-TGTGGTCACGCTGGGGGGGGGGGCCCGAGTGAGCACG-3′) (SEQ ID NO:8). The resulting construct, pHLP19, is 7327 bp in length.

### pLadeno1 Accessory Function Vector Construction

The pLadeno 1, accessory function vector is described in detail in U.S. Pat. No. 6,004,797, herein incorporated by reference in its entirety. To summarize, pLadeno 1 containing adenovirus VA RNA, E4 and E2a gene regions, was assembled by cloning adenovirus type-5 genes into a custom polylinker that was inserted between the PvuII sites of pBSII s/k-. More particularly, a double stranded oligonucleotide polylinker encoding the restriction enzyme sites SalI-XbaI-EcoRV-SrfI-BamHI (5′-GTCGACAAATCTAGATATCGCCCGGGCGGATCC-3′) (SEQ ID NO:9) was ligated to the 2513 bp PvuII vector fragment of pBSII s/k- to provide an assembly plasmid. The following fragments containing adenovirus type-5 genes or gene regions were then obtained from the pJM17 plasmid (the pJM17 plasmid described in detail in McGrory et al. (1988) Virology 163:614-617): the 1,724 bp SalI-HinDIII VA RNA-containing fragment (corresponding to the nucleotides spanning positions about 9,831 to about 11,555 of the adenovirus type-2 genome—the complete adenovirus type-2 genome available under GenBank Accession Number NC_{—}001405); the 5,962 bp SrfI-BamHI E2a-containing fragment (corresponding to the nucleotides spanning positions about 21,606 to about 27,568 of the adenovirus type-2 genome); and the 3,669 bp HphI-HinDIII E4-containing fragment (corresponding to the nucleotides spanning positions about 32,172 to about 36,841 of the adenovirus type-2 genome). AnXbaI site was added to the HphI end of the E4-containing fragment by cloning the 3,669 bp HphI-HinDIII fragment into the HpaI site of cloning vector, and then excising the fragment with XbaI and HinDIII (partial digestion). The 5,962 E2a-containing fragment was cloned between the SrfI and BamHI sites of the assembly plasmid, and the 1,724 bp VA RNA-containing fragment and the modified 3,669 bp E4-containing fragments were joined by their common HinDIII ends and ligated between the SalI and XbaI sites of the assembly plasmid to obtain the pLadeno 1 construct.

### Recombinant AAV-LacZ Vector Construction

The recombinant AAV-LacZ vector was constructed as follows: A 2.7-kb KasI-EarI fragment from pUC119 (GenBank Accession No. U07650) was blunted and ligated to a multiple cloning sequence containing the following restriction enzyme sites (5′-NotI-MluI-SnaBI-AgeI-BstBI-BssHII-NcoI-HpaI-BspEI-PmlI-RsrII-NotI-3′). The following fragments were successively cloned into the SnaBI site, a BstBI-BstBI fragment from the human growth hormone first intron was inserted into the BstBI site, the lacZ gene was ligated into the BssHII site, and HpaI-BamHI fragment of the simian virus 40 (SV40) polyadenylation signal sequence was cloned into the HpaI site. The resulting NotI-NotI expression cassette was inserted between the AAV 145-bp inverted terminal repeats of a pUC-based plasmid.

### Recombinant AAV-LacZ Virion Production

Recombinant AAV-LacZ virions were produced using a triple transfection method described in U.S. Pat. Nos. 6,001,650 and 6,004,797, supra. To summarize, cells from the stable human cell line, 293 (readily available through, e.g., ATCC under Accession Number CRL1573), were plated in eight 10-cm tissue culture dishes at 1×10⁶ cells at 37° C. to reach 90% confluency over a period of from about 24 to 48 hours prior to transfection.

Transfections were carried out using the calcium phosphate method. Specifically, at 1 to 4 hours prior to transfection, the medium in the tissue culture plates was replaced with fresh Dulbeco's Modified Eagles Medium (DMEM)/F12 (GIBCO, BRL) containing 10% fetal calf serum, 1% penicillin/streptomycin, and 1% glutamine. A total of 10 μg each of DNA from the three vectors, pHLP19, pLadeno 1, and rAAV-TH were added to 1 mL of sterile 300 mM CaCl₂, which was then added to 1 mL of sterile 2×HBS solution (formed by mixing 280 mM NaCl, 50 mM HEPES buffer, 1.5 mM Na₂HPO₄ and adjusting the pH to 7.1 with 10 M NaOH) and immediately mixed by gentle inversion. The resultant mixture was pipetted immediately into the 10 cm plates of 90% confluent 293 cells (in 10 mL of the above-described culture medium) and swirled to produce a homogeneous solution. The plates were transferred to a 5% CO₂ incubator and cultured at 37° C. for approximately 5 hours without disturbing. After transfection, the medium was removed from the plates, and the cells washed once with sterile Phosphate buffered saline (PBS). New culture medium was added and the cells were incubated at 37° C. for approximately 72 hours.

The cells were then collected, media was removed by centrifugation (1000×g for 10 min.), and a 1 mL lysate (cells lysed in Tris buffer—10 mM Tris 150 mM NaCl, pH 8.0) was produced using 3 freeze/thaw cycles (alternating between dry ice-ethanol and 37° C. water baths). The lysates were made free of debris by centrifugation (12,000×g for 10 min).

Recombinant AAV-LacZ virions were then purified by two sequential continuous cesium chloridate gradient ultracentrifugations. Recombinant AAV-LacZ titer was determined by quantitative dot-blot hybridization of DNAseI-treated recombinant AAV-LacZ stocks.

### EXAMPLE 2

### PERIOSTEAL CELL HARVEST AND CULTURE

Periosteum measuring 5×15 mm was harvested from the medial side of the tibia of a six-week-old Japanese white rabbit. The harvested periosteum was cut with a sterilized blade in small pieces and then the pieces were digested with 5 mL of 0.05% trypsin-EDTA (Sigma, St. Louis, Mo.) for 20 min. After centrifuging the resultant solution at 1000 rpm for 5 min, the solution was removed and then subjected to further digestion with 0.25% collagenase (Worthington Biochemical Corp., Lakewood, N.J.) for 3 hr at 37° C. with mild shaking in a waterbath. About 30,000 cells were obtained from one piece of the periosteum.

The periosteal cells were then plated in Dulbecco's Modified Eagle Medium (DMEM, Sigma, St. Louis Mo.) containing penicillin (0.05 units/mL) and streptomycin (0.05 μg/mL) (Gibco BRF, UK) supplemented with 10% fetal bovine serum and then incubated at 37° C. in an atmosphere of 5% CO₂ in air. Confluent cells were harvested by treatment with 0.05% trypsin-EDTA and plated in two 60-mm diameter plates.

### EXAMPLE 3

### RECOMBINANT AAV-LacZ VIRION TRANSDUCTION

Subconfluent periosteal cell cultures were washed with PBS, and 0.5 mL of rAAV-LacZ (10⁸ vector genomes/mL) in DMEM (without FBS) was added to each cell culture plate and incubated for 1 hr as before. After incubation, DMEM containing 10% FBS was added to each plate and the plates were incubated as before for 1 week.

### EXAMPLE 4

### PERIOSTEAL CELLS GROWN IN COLLAGEN GEL

One week after rAAV-LacZ transduction, periosteal cells were harvested and mixed with 80 μL of 0.25% collagen gel (DME-02 Koken Cellgen, Japan) at a density of 4×10⁶/mL and cultured for 2 days as before. After culture, periosteal cells were used for subsequent transplantation to a cartilage defect in a rabbit model.

### EXAMPLE 5

### EX VIVO TRANSPLANTION OF PERIOSTEAL CELLS

Ten Japanese white rabbits were anesthetized by intravenous injection of sodium pentobarbital. A full-thickness defect (diameter 5 mm, depth 3 mm) was made at the femoral patellar groove by drilling and then filled with the collagen gel containing rAAV-LacZ-transduced periosteal cells. Another periosteum was harvested from the tibia to create periosteal patches, which were placed over the collagen gel matrix. Periosteum patches were fixed at the edge of the cartilage defect with 5-0 nylon. In the other knee, a sham operation was performed as a control, the defect was filled with cell-free collagen or non-transduced periosteal cells.

### EXAMPLE 6

### LacZ EXPRESSION IN PERIOSTEAL CELLS

LacZ expression was detected in cultured periosteal cells, periosteal cell-containing collagen matrix, and transplanted periosteal cells by first fixing the periosteal cells in solution containing 2% formaldehyde and 0.2% glutaraldehyde in PBS and then staining with 5-bromo-4-chloro-3-indolyl-beta-D-galactoside (X-gal, 1 mg/mL, containing K₄Fe(CN)₆/3H₂O, K₃Fe(CN)₆, MgCl₂, and Na deoxycholate in PBS) directly. The LacZ expression on each specimen was assayed from 3 days to 12 weeks after rAAV-LacZ transduction.

### Cell Culture Data

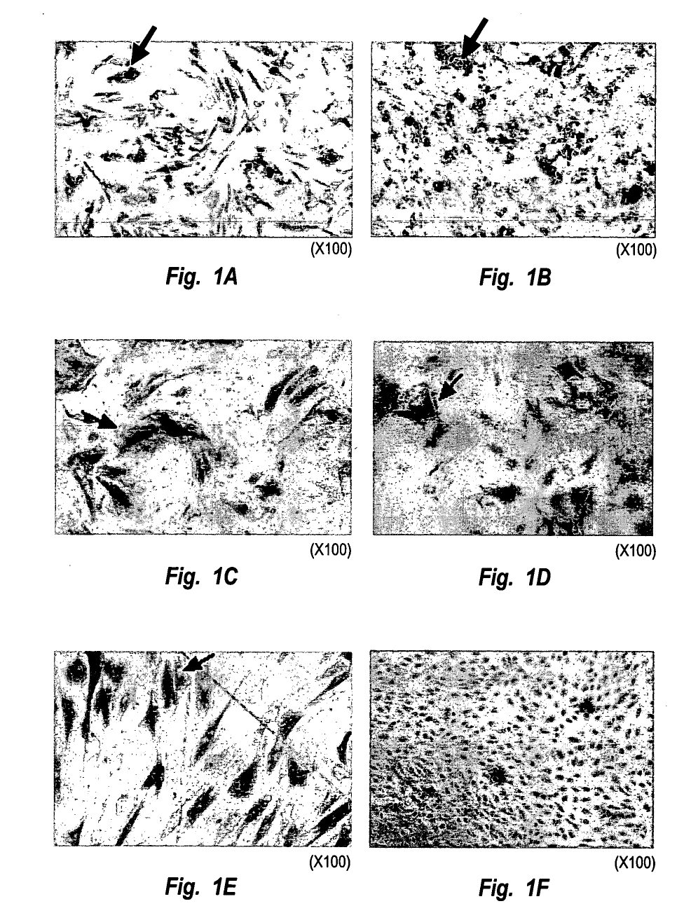

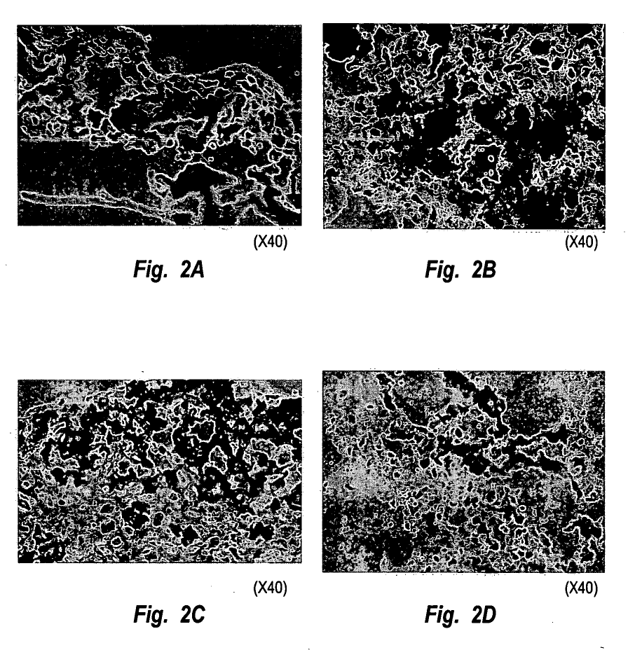

The number of LacZ-positive periosteal cells and total periosteal cells were averaged at four sights under light microscopy at X100 magnification (FIG. 1). Mean percentage of LacZ-positive periosteal cells per total periosteal cells was calculated using data from three independent experiments. 54.2±10.2% of the total number of periosteal cells were LacZ positive 3 days post rAAV-LacZ transduction (FIG. 2A). After one week, 68.2±3.8% of the periosteal cells were LacZ positive (FIG. 2B). LacZ positive periosteal cells remained consistently blue at two and four weeks (FIG. 2C and FIG. 2D) post transduction. After twelve weeks post-transduction, 53.2±11.7% of the total number of periosteal cells were LacZ positive.

### Collagen Matrix Data

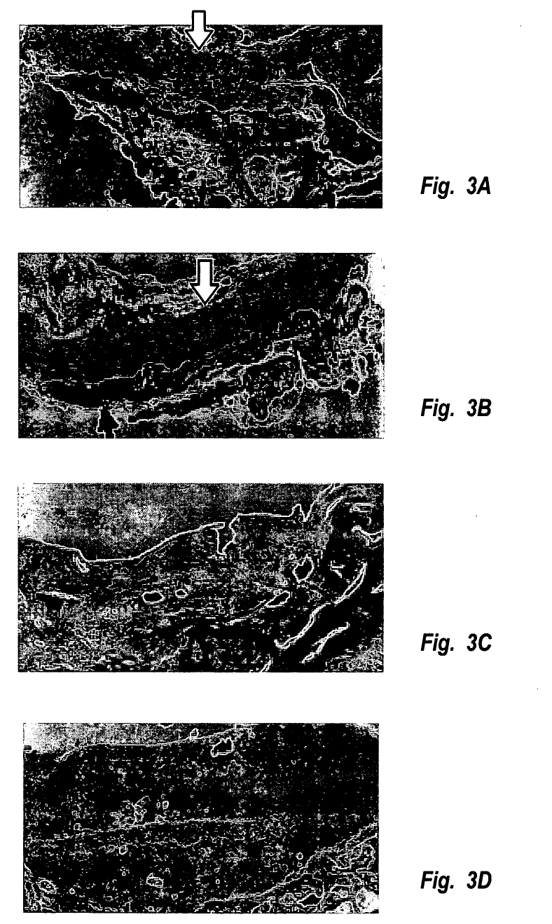

LacZ positive periosteal cells were observed in all four specimens (FIG. 3A-3D). Staining lasted for at least four weeks, the duration of the collagen matrix experiment.

### Transplantation Data

In eight of ten Japanese white rabbits receiving a transplant of collagen matrix containing rAAV-LacZ-transduced periosteal cells, strong expression of LacZ was detected. One week after transplantation, LacZ positive periosteal cells were detected under the periosteum patch (FIG. 4A) and were still evident after two weeks (FIG. 4B). In both the cell-free collagen gel and the collagen gel specimen containing non-transduced periosteal cells, LacZ positive periosteal cells were not detected (FIG. 4C and 4D).

### EXAMPLE 7

### RECOMBINANT AAV-TGF-BETA1 VIRION TRANSDUCTION

Recombinant AAV virions comprising the transforming growth factor beta (TGF-β) gene are made as in Example 1. Specifically, the TGF-β1 gene (GenBank Accession No. XM_{—}008912) is used to construct a rAAV-TGF-β1 vector for subsequent experimental use. Using standard recombinant techniques that are well known in the art, the rAAV-TGF-β1 vector is constructed by excising the LacZ gene from the rAAV-LacZ vector and inserting the TGF-β1 gene in its place. Periosteal cells are harvested as in Example 2 and transduced in vitro as in Example 3 and grown in cell culture or grown in a collagen matrix as in Example 4. Transduced periosteal cells are then transplanted onto a Japanese white rabbit as in Example 5. Gene expression is detected using an enzyme-linked-immunosorbent assay specific for TGF-β1 (Promega, Madison Wis.). After one week and two weeks post-transplantation of transduced periosteal cells, visual inspection is used to identify and quantify new articular cartilage growth.

We claim:

1. A method of delivering a heterologous gene to a periosteal cell of a mammal, comprising:
a) providing recombinant adeno-associated virus (rAAV) virions, wherein said rAAV virions comprise said heterologous gene;
b) contacting said periosteal cell with said rAAV virions, wherein said contacting results in transduction of said periosteal cell by said rAAV virions; and
c) expressing said heterologous gene in said periosteal cell.

2. A method of expressing a heterologous gene in a mammal, comprising:
(a) providing recombinant adeno-associated virus (rAAV) virions, wherein said rAAV virions comprise said heterologous gene;
(b) contacting a periosteal cell with said rAAV virions, wherein said contacting results in transduction of said periosteal cell by said rAAV virions;
(c) delivering said periosteal cell to said mammal; and
(d) expressing said heterologous gene.

3. The method of claim 2, wherein said periosteal cell is contained within a matrix.

4. The method of claim 3, wherein said matrix is composed of collagen.

5. The method of claim 4, wherein said collagen is type II collagen.

6. The method of claim 2, wherein said mammal has a skeletal disorder.

7. The method of claim 6, wherein said skeletal disorder is an articular cartilage defect.

8. The method of claim 7, wherein said articular cartilage defect is osteoarthritis.


SEQUENCE LISTING (9 sequences)

SEQ ID NO: 1 - DNA, 30 bp, Artificial Sequence
  synthetic oligonucleotide
gctcggtacccgggcggaggggtggagtcg

SEQ ID NO: 2 - DNA, 30 bp, Artificial Sequence
  synthetic oligonucleotide
taatcattaactacagcccggggatcctct

SEQ ID NO: 3 - DNA, 24 bp, Artificial Sequence
  synthetic oligonucleotide
ggtttgaacgagcgctcgccatgc

SEQ ID NO: 4 - DNA, 42 bp, Artificial Sequence
  synthetic oligonucleotide
cgcgccgatatcgttaacgcccgggcgtttaaacagcgctgg

SEQ ID NO: 5 - DNA, 42 bp, Artificial Sequence
  synthetic oligonucleotide
cgcgccagcgctgtttaaacgcccgggcgttaacgatatcgg

SEQ ID NO: 6 - DNA, 7 bp, adeno-associated virus 2
tatttaa

SEQ ID NO: 7 - DNA, 7 bp, Artificial Sequence
  synthetic sequence
ggggggg

SEQ ID NO: 8 - DNA, 37 bp, Artificial Sequence
  synthetic oligonucleotide
tgtggtcacgctgggggggggggcccgagtgagcacg

SEQ ID NO: 9 - DNA, 33 bp, Artificial Sequence
  synthetic oligonucleotide
gtcgacaaatctagatatcgcccgggcggatcc